(19) United States
(12) Patent Application Publication
(10) Pub. No.: US 2025/0216369 A1
(43) Pub. Date: Jul. 3, 2025

(54) Gas Detection, Identification, and Quantification Systems and Devices for Medical Applications

(71) Applicant: Becton, Dickinson and Company, Franklin Lakes, NJ (US)

(72) Inventors: Mildred Calistri-Yeh, Killingworth, CT (US); Vasudha Kotia, Bangalore (IN); Klaus Heinrich Hoerauf, Mahwah, NJ (US); Ajinkya Madhukar Joglekar, Edgewater, NJ (US)

(21) Appl. No.: 18/844,999

(22) PCT Filed: Mar. 22, 2023
(86) PCT No.: PCT/US23/15845

§ 371 Date: Sep. 9, 2024

(30) Foreign Application Priority Data
Mar. 23, 2022 (IN) 202211016180

(51) Int. Cl. G01N 33/00; A61M 16/00

(52) U.S. Cl. CPC G01N 33/0047; A61M 16/0051; A61M 16/0087; A61M 16/024; G01N 33/0027; G01N 33/0031; G01N 33/0073; A61M 2230/437

(57) ABSTRACT

A system for detection of volatile organic compounds (VOCs) in a medical facility includes at least one VOC sensor configured to sense VOCs in ambient air of the medical facility; and a controller electrically connected to the at least one VOC sensor. The controller is configured to: receive and process signals from the at least one VOC sensor; determine a concentration of at least one VOC in ambient air proximate to the at least one VOC sensor based on the received and processed signals; compare the determined concentration of the at least one VOC to a threshold value to determine when the detected concentration exceeds the threshold value; and provide an alarm when the detected concentration of the at least one VOC exceeds the threshold value.

### CROSS-REFERENCE TO RELATED APPLICATION

The present application claims priority to Indian patent application Ser. No. 20/221,1016180, filed Mar. 23, 2022, the entire disclosure of which is hereby incorporated by reference in its' entirety.

### BACKGROUND OF THE INVENTION

### Field of the Invention

The present invention relates generally to gas sensors and detectors and, in particular, to sensing devices and systems for detecting, identifying, and/or quantification of compounds and chemicals, especially volatile organic compounds (VOCs), for medical applications and/or in medical facilities.

### Description of the Related Art

In medical facilities, patients are routinely treated with chemicals that interact with the body in order to treat illness or to prepare the body for interventional therapies. In some cases, these chemicals are selected to kill cells (e.g., chemicals for chemotherapy or radio-isotope therapy) or to cause grogginess/sleepiness (e.g., gaseous or intravenous anesthesia agents) for patients undergoing medical treatments. In addition, medical devices used for patient treatments are often treated with toxic chemicals for sterilization prior to use. Pharmacists, medical professionals, other staff, and family members of the patients should not be exposed to unsafe concentrations of these chemicals and substances because prolonged exposure to such substances, especially in the occupational setting, can cause long-term harm.

One specific problem, which can occur in operating rooms, is caused by inhaled anesthesia. Most commonly-used anesthesia agents comprise halogenated hydrocarbons, such as sevoflurane, enflurane, desflurane, halothane, or isoflurane. These anesthesia agents are typically used in combination with nitrous oxide to produce surgical levels of anesthesia. Occupational exposure issues due to long-term exposure to halogenated anesthetic chemicals can include acute headaches, drowsiness, or difficulties with judgement and coordination, as well as chronic adverse reproductive effects, cancer, liver damage and even death. There are also issues with exposure to nitrous oxide. For example, prolonged exposure to nitrous oxide can cause acute conditions, such as lightheadedness or shortness of breath, as well as chronic conditions, such as reduced fertility, spontaneous abortion, neurologic disease, or renal and/or liver disease.

Anesthetic gases and vapors that leak into the surrounding room are considered waste anesthetic gases (WAGs). At present, more than 250,000 healthcare workers in the United States may have been exposed to WAGs and are at risk of developing adverse health effects. In some cases, waste gases may escape from an anesthesia machine into the operating room through leaks in various components of the machine and other medical accessories. Parts that are subject to leaks can include gas canisters, valves, high and low pressure connections, defective rubber or plastic tubing, hoses, and ventilator bellows. Leaks may also occur due to simple human errors. For example, leaving gas flow control valves open, keeping vaporizers on after use, spilling liquid anesthetics, or placing poorly fitting face masks on patients can all cause waste anesthesia gases to escape the machine and enter the room.

Waste anesthesia monitoring is conducted to maintain staff safety in operating rooms and other medical facilities to comply with standards set forth by, for example, the National Institute for Occupational Safety and Health (NIOSH), the Occupational Safety and Health Administration (OSHA), the Department of Health and Human Services (DHHS), The Joint Commission (TJC), and other regulatory agencies. However, most WAG monitoring processes currently in use require medical facilities to send gas samples to independent laboratories for testing. For example, medical facility staff may wear waste anesthetic gas badges that collect gas samples over time. The badges and collected samples are sent to a laboratory or to a separate analyzer for testing. A normal waiting time from sampling to results can be two weeks or longer. Such extensive delays between sampling and results means that unsafe conditions, such as gas leaks or repeated human errors, cannot be quickly corrected to limit exposure of the medical staff to hazardous gasses.

In view of the delays in current testing processes, there is a need for sensors for detecting WAGs and VOCs more quickly, desirably in real time. Also, there is a need to provide timely alarms to protect medical professionals from effects of inhaling the WAGs and VOCs. The devices and systems of the present disclosure are configured to address these issues.

### SUMMARY OF THE INVENTION

According to an aspect of the present disclosure, a system for detection of volatile organic compounds (VOCs) in a medical facility includes at least one VOC sensor configured to sense VOCs in ambient air of the medical facility; and a controller electrically connected to the at least one VOC sensor. The controller is configured to: receive and process signals from the at least one VOC sensor; determine a concentration of at least one VOC in ambient air proximate to the at least one VOC sensor based on the received and processed signals; compare the determined concentration of the at least one VOC to a threshold value to determine when the detected concentration exceeds the threshold value; and provide an alarm when the detected concentration of the at least one VOC exceeds the threshold value.

Non-limiting illustrative examples of embodiments of the present disclosure will now be described in the following numbered clauses.

Clause 1: A system for detection of volatile organic compounds (VOCs) in a medical facility, the system comprising: at least one VOC sensor configured to sense VOCs in ambient air of the medical facility; and a controller electrically connected to the at least one VOC sensor configured to: receive and process signals from the at least one VOC sensor; determine a concentration of at least one VOC in ambient air proximate to the at least one VOC sensor based on the received and processed signals; compare the determined concentration of the at least one VOC to a threshold value to determine when the detected concentration exceeds the threshold value; and provide an alarm when the detected concentration of the at least one VOC exceeds the threshold value.

Clause 2: The system of clause 1, wherein the medical facility comprises at least one of a hospital, operating room, recovery room, intensive care unit, out-patient medical office, dental office, ambulance, or veterinary clinic.

Clause 3: The system of clause 1 or clause 2, wherein the at least one VOC sensed by the at least one VOC sensor comprises a halogenated hydrocarbon.

Clause 4: The system of any of clauses 1-3, wherein the at least one VOC sensed by the at least one VOC sensor comprises at least one of sevoflurane, enflurane, desflurane, halothane, isoflurane, nitrous oxide, indole, methanol, undecene, ethylacetate, 2,4-dimethyl-1-heptane, butanone, benzaldehyde, dimethylcyclohexanol, isovaleric acid, 2-pentanol, methylquinazoline, or methyl butyraldehyde.

Clause 5: The system of any of clauses 1-4, wherein the at least one VOC sensed by the at least one VOC sensor is a sterilization gas, such as ethylene oxide (EtO) or hydrogen peroxide.

Clause 6: The system of any of clauses 1-5, wherein the at least one VOC sensed by the at least one VOC sensor comprises VOCs emitted from hazardous drugs to be delivered to a patient, such as chemotherapy drugs.

Clause 7: The system of any of clauses 1-6, further comprising a housing, wherein the at least one VOC sensor and the controller are enclosed within and/or mounted to the housing.

Clause 8: The system of clause 7, wherein the housing comprises a clip for mounting the housing to at least one of a patient bed, surgical curtain or drape, medical cart or stand, medical device (e.g., anesthesia gas equipment), an anesthesia waste gas scavenger, pharmaceutical workstation, ventilated hood, or pharmaceutical storage area.

Clause 9: The system of clause 7 or clause 8, wherein the housing is a wearable housing or is configured to be mounted to a portable electronic device, such as a smart phone or computer tablet

Clause 10: The system of any of clauses 1-9, further comprising an audio/visual feedback device electrically connected to the controller, wherein the controller is configured to cause the feedback device to emit the alarm to a user.

Clause 11: The system of clause 10, wherein the audio/visual feedback device comprises at least one of a speaker, visual display, or light emitter.

Clause 12: The system of clause 10 or clause 11, wherein the controller is further configured to cause the audio/visual feedback device to display an indication of the concentration of the at least one VOC on the visual display of the audio/visual feedback device.

Clause 13: The system of clause 12, wherein the indication comprises a numerical value for the determined concentration displayed on the visual display.

Clause 14: The system of any of clauses 1-13, further comprising a wireless transmitter electrically coupled to the controller, wherein the controller is configured to cause the wireless transmitter to transmit the alarm and/or information sensed by the at least one VOC sensor to a remote device, system, or computer network.

Clause 15: The system of any of clauses 1-14, wherein the at least one VOC sensor comprises at least one of a metal oxide-based sensor, a sensor comprising graphene or a graphene derivative, or an infrared detector.

Clause 16: The system of clause 15, wherein the infrared detector comprises a FTIR gas analyzer sensor or a photoacoustic infrared spectroscopy sensor.

Clause 17: The system of any of clauses 1-16, wherein the at least one sensor comprises a biosensor configured to detect peptide molecules based on optical properties of the molecules.

Clause 18: The system of any of clauses 1-17, further comprising a humidity and/or temperature sensor.

Clause 19: The system of clause 18, wherein processing the signals from the at least one VOC sensor comprises normalizing the received signals for at least one of humidity or temperature based at least in part in information sensed by the humidity and/or temperature sensor.

Clause 20: The system of any of clauses 1-19, wherein processing the received signals comprises distinguishing between signals representative of the at least one VOC and signals representative of cleaning chemicals (such as isopropyl alcohol, hydrogen peroxide, or bleach) used during medical procedures.

Clause 21: The system of any of clauses 1-20, wherein processing the received signals comprises removing artifacts in the received signals representative of chemicals other than the at least one VOC being sensed from the signals.

Clause 22: The system of any of clauses 1-21, further comprising an anesthesia machine, wherein the at least one VOC sensor is positioned to detect ambient air proximate to an exhalation portion of the anesthesia machine.

Clause 23: The system of clause 22, wherein the anesthesia machine comprises: at least one gas cylinder, at least one gas pressure and/or flow regulator, a vaporizer, a delivery/breathing circuit, at least one of a pressure sensor, airflow sensor, temperature sensor, oxygen sensor, or CO₂ sensor fluidly connected to the delivery/breathing circuit for monitoring air inhaled or exhaled by the patient, a scavenging system, and a face mask.

Clause 24: The system of clause 22 or clause 23, wherein the controller is further configured to compare the determined concentration of the at least one VOC to a normal value for a patient prior to commencing anesthesia treatment; and provide an indication that a patient's breathing has returned to normal when the concentration is below or within a predetermined amount of the normal value for the patient.

Clause 25: The system of clause 24, wherein the indication further comprises an instruction to remove the mask from the patient's face.

Clause 26: The system of any of clauses 1-25, wherein the at least one VOC sensor comprises an array of a plurality of VOC sensors, and wherein the at least one processor is configured to process signals received from the plurality of VOC sensors to determine a signature pattern comprising concentrations of the VOCs measured by the plurality of VOC sensors.

Clause 27: The system of any of clauses 1-26, further comprising computer memory electrically connected to the controller, the memory comprising previously-determined signature patterns for a plurality of VOC compounds, and wherein the controller is further configured to compare the determined signature pattern to the previously-determined signature patterns to identify the VOC compound sensed by the sensor array.

Clause 28: The system of clause 27, wherein the controller is further configured to update the signature patterns stored on the computer memory for the identified VOC compound based on the signals sensed by the VOC sensors of the sensor array.

Clause 29: The system of any of clauses 1-28, further comprising a calibration sensor for calibrating the at least one VOC sensor.

Clause 30: The system of clause 29, wherein the at least one VOC sensor and the calibration sensor are fluidly connected together, such that the ambient air passes from the calibration sensor to the at least one VOC sensor.

Clause 31: The system of clause 30, further comprising at least one flow tube defining an air pathway between the calibration sensor and the at least one VOC sensor.

Clause 32: The system of clause 31, further comprising a fan or pump fluidly connected to the air pathway configured to draw ambient air into and through the air pathway.

### BRIEF DESCRIPTION OF THE DRAWINGS

FIG. 1 is a schematic drawing of a system for VOC detection for a medical facility, according to an aspect of the present disclosure.

FIG. 2 is a schematic drawing of a portable VOC detection device, according to an aspect of the present disclosure.

FIGS. 3A-3C are schematic drawings of a wearable VOC detection device, according to an aspect of the present disclosure.

FIG. 4 is a schematic drawing of an anesthesia machine including a VOC sensor, according to an aspect of the present disclosure.

FIG. 5 is a schematic drawing of another example of a VOC detecting device, according to an aspect of the present disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

As used herein, the singular form of “a”, “an”, and “the” include plural referents unless the context clearly dictates otherwise.

All numbers and ranges used in the specification and claims are to be understood as being modified in all instances by the term “about”. By “about” is meant plus or minus twenty-five percent of the stated value, such as plus or minus ten percent of the stated value. However, this should not be considered as limiting to any analysis of the values under the doctrine of equivalents.

The present disclosure is directed to sensors 12, 112, 212, 312 for sensing, detecting, identifying, and/or quantifying a concentration of a volatile organic compound (VOC) in ambient air of a medical facility or in other gaseous environments. As used herein, a volatile organic compound (VOC) refers to various compounds with a high vapor pressure and low water solubility. Most VOCs are human-made chemicals that are used and produced during manufacture of pharmaceuticals, as well as during manufacture of paints, coatings, and refrigerants. In some examples, VOCs can also include industrial solvents, such as trichloroethylene, fuel oxygenates, such as methyl tert-butyl ether (MTBE), or by-products produced by chlorination in water treatment, such as chloroform. Other VOCs are components of petroleum fuels, hydraulic fluids, paint thinners, and dry cleaning agents. VOCs associated with medical uses include, for example, sevoflurane, enflurane, desflurane, halothane, isoflurane, nitrous oxide, indole, methanol, undecene, ethylacetate, 2,4-dimethyl-1-heptane, butanone, benzaldehyde, dimethylcyclohexanol, isovaleric acid, 2-pentanol, methylquinazoline, or methyl butyraldehyde. In some examples, VOCs can include halogenated hydrocarbon compounds, such as compounds used in anesthesia agents. The VOCs can also include sterilization chemicals, such as ethylene oxide (EtO).

In some examples, the detection devices, systems, and VOC sensors 12, 112, 212, 312 of the present disclosure can be small in size (i.e., portable, able to integrate easily in clinical setting), easy to handle, able to take multiple measurements at sufficiently high frequency to give results frequently, require a short amount of time to make a measurement (seconds to minutes), provide reproducible results, and can be used many times before repair or replacement parts are required. Further, as described in further detail herein, the VOC sensors 12, 112, 212, 312 and devices of the present disclosure can be linked with gas sampling protocols and data interpretation routines to monitor gas accumulation and provide alerts, alarms, warnings, and notifications at appropriate times.

In some examples, the VOC sensors 12, 112, 212, 312 of the present disclosure can be configured to identify a particular VOC of interest in the ambient air and/or to determine a concentration of one or more VOCs in the ambient air. The devices and systems of the present disclosure can also be configured to monitor a concentration of the VOC in ambient air over time and automatically provide, substantially in real time, an alarm or alert to a user, such as a medical professional performing a procedure or to bystanders (e.g., other medical facility staff or family members of a patient), about potential exposure to a VOC and/or when a concentration of the VOC in ambient air exceeds a predetermined threshold amount.

In some examples, the VOC sensors 12, 112, 212, 312 of the present disclosure can be integrated in a stand-alone device to provide VOC detection, concentration measurements, alerts, and warnings. For example, the stand alone device can be a portable and/or hand-held device or a wearable device used in clinical settings. Workers can carry the detection devices with them during performance of any activities in proximity to VOC sources. Alternatively, workers can place the portable VOC detection devices in convenient locations, such as resting on a table or attached to an object by a clip, to detect VOCs in proximity to a worker's workspace.

In other examples, the VOC sensors 12, 112, 212, 312 can be integrated into equipment that handles VOC gases, such as gas delivery devices or anesthesia machines, to monitor and provide safeguards for identifying when VOCs accumulate to an unsafe amount in proximity to the gas equipment. For example, VOC sensors 12, 112, 212, 312 can be positioned on a return gas or exhalation side of an anesthesia machine to ensure that the patient's exhaled gases have returned to normal levels before a patient's anesthesia mask is removed.

In other examples, the VOC sensors 12, 112, 212, 312 and devices can be integrated into a central sterilization area of a medical facility to detect a concentration of sterilization gasses in ambient air of the sterilization area. Ethylene oxide (EtO) gas, which is used in the sterilization of the medical devices and other materials, is a carcinogenic agent. Acute exposure to EtO may cause headache, nausea, vomiting, diarrhea, breathing difficulty, drowsiness, weakness, exhaustion, eye and skin burns, frostbite, and/or reproductive defects. The VOC sensors 12, 112, 212, 312 and devices of the present disclosure can be configured to detect EtO concentration and provide appropriate alarms for workers before EtO concentration reaches unsafe levels.

### VOC Detection System

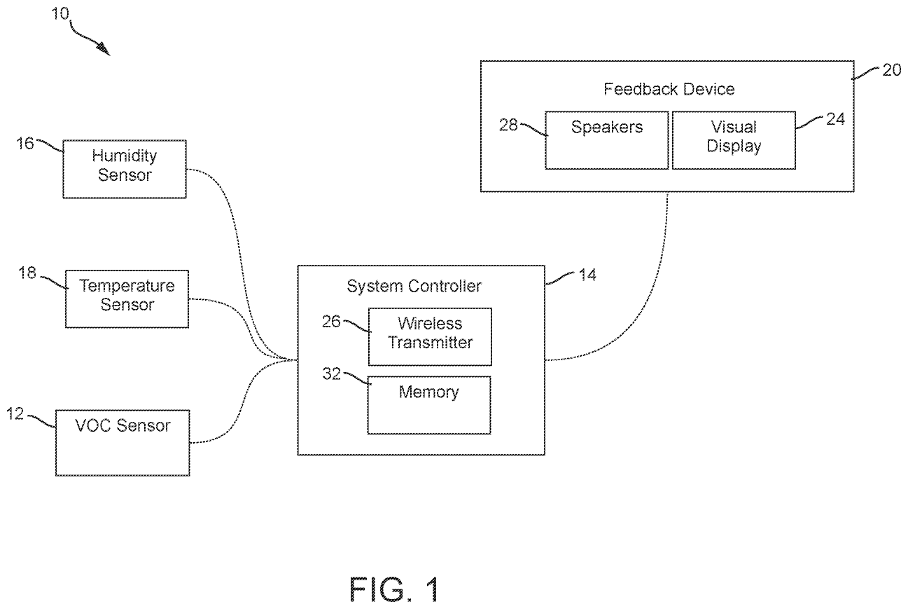

With reference to FIG. 1, an exemplary system 10 for detection of VOCs in a medical facility comprises a VOC sensor 12 configured to sense VOCs in ambient air of the medical facility and a controller 14 electrically connected to the VOC sensor 12. The medical facility can be any building, vehicle, or location including medical equipment or containers that contain VOCs. For example, the medical facility can be a hospital, operating room, recovery room, intensive care unit, out-patient medical office, dental office, ambulance, or veterinary clinic. Generally, the medical facility is an indoor or enclosed space in which medical professionals, such as physicians, nurses, medical technicians, and other trained individuals, work in proximity and/or may be exposed to pharmaceuticals and other chemicals containing VOCs.

The VOC sensors 12 can include a variety of different sensor designs configured to output electrical signals in response to and/or representative of a concentration of the VOC in ambient air in proximity to the VOC sensor 12. In some examples, the VOC sensors 12 comprise reactive layers or portions that undergo changes in physical and/or chemical properties in a presence of certain VOCs. The VOC sensors 12 can also include electric circuitry for detecting the changes in the reactive layers or portions caused by the VOCs. For example, some VOC sensors 12 include optical circuitry, such as infrared light or visual light detectors, for detecting changes in appearance or radiation emission of the reactive layers or portions of the VOC sensors 12. Alternatively or in addition, some VOC sensors 12 can include electrical circuitry for detecting changes in electrical properties, such as impedance, conductance, or resistance, of the reactive layers or portions of the sensors 12. In some examples, the VOC sensors 12 can comprise metal oxide-based sensors, sensors comprising graphene or a graphene derivative, hot-wire anemometers, infrared detectors (e.g., an FTIR gas analyzer sensor or a photoacoustic infrared spectroscopy sensor), or biosensors, such as biosensors comprising peptide molecules that react with VOCs.

With continued reference to FIG. 1, the VOC sensors 12 can be configured to be positioned at different selected locations of a medical facility and/or to be placed on or mounted to different medical devices in the medical facility. For example, the VOC sensors 12 can be positioned on objects commonly found in medical facilities, such as attached to a surgical/patient bed, operating tables, surgical curtains or drapes, medical carts, or to medical devices, such as an anesthesia machine. In other examples, the VOC sensors 12 can be mounted or attached to a pharmaceutical workstation, ventilation hood, or storage area to detect VOCs created during production, mixing, or reconstitution of pharmaceutical compositions, in particular to detect gasses created during mixing and/or reconstitution of hazardous drugs. In other examples, as previously described, the VOC sensors 12 and/or other components of the system 10 can be wearable. For example, the VOC sensors 12 can be connected to or embedded in a watch, wristband, necklace, pendant, work badge, or other wearable items, which can be worn by a clinician, pharmacist, or other medical professionals while performing medical activities in proximity to a VOC source. In other examples, the VOC sensors 12 and/or other components of the system 10 can be mounted to a portable electronic device, such as a smart phone or computer tablet.

The controller 14 of the system 10 can be a computer microprocessor or another computing device electrically connected to the VOC sensor 12 configured to generate alarms, alerts, and/or notifications based on signals detected by the VOC sensor 12. For example, the controller 14 can be configured to receive and process signals from the VOC sensor 10; determine a concentration of a particular or selected VOC in ambient air proximate to the VOC sensor 12 based on the received and processed signals; compare the determined concentration of the particular or selected VOC to a threshold value to determine when the detected concentration exceeds the threshold value; and provide an alarm when the detected concentration of the VOC exceeds the threshold value.

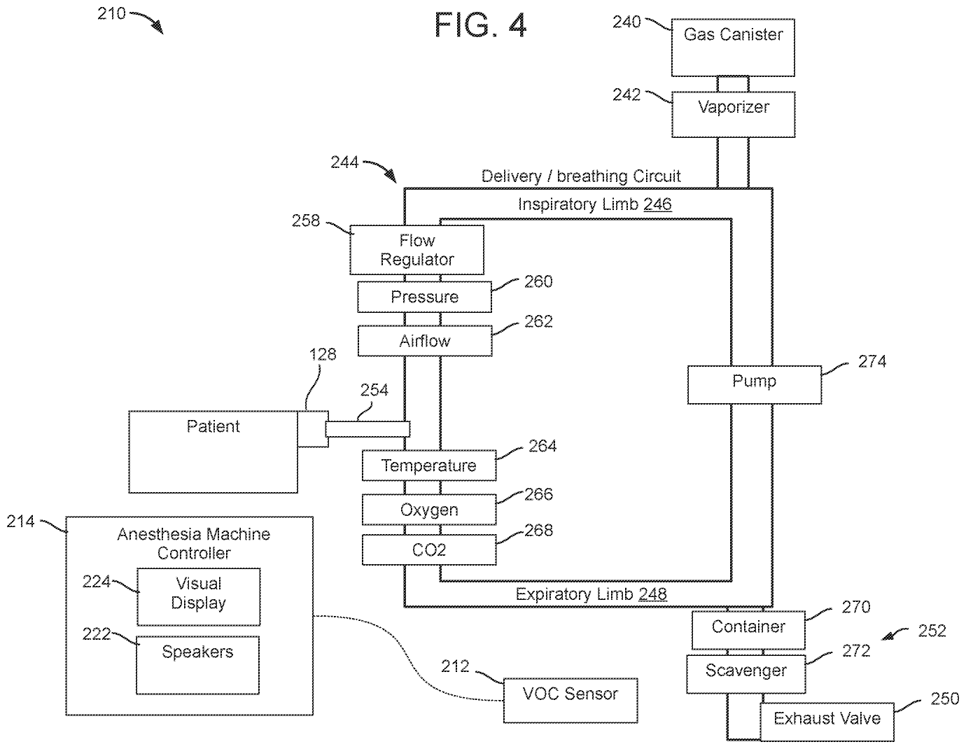

The system 10 can also include additional sensors for sensing other information about the ambient air and/or environment where the VOC sensor 12 is located. In some examples, as described in further detail herein, information from such environmental sensors can be used for calibrating the VOC sensor 12 and/or for confirming accuracy of measurements obtained by the VOC sensor 12. For example, the system 10 can include a humidity sensor 16 and/or a temperature sensor 18 for detecting humidity and temperature of the ambient air. When used with an anesthesia machine (shown in FIG. 4), the system 10 can also include sensors for measuring gas inhaled or exhaled by the patient including, for example, pressure sensors, airflow sensors, oxygen sensors, and/or carbon dioxide sensors.

With continued reference to FIG. 1, the system 10 can also include an external alarm or feedback device 20 for providing alarms, alerts, notifications, and other information to a user or bystanders. In some examples, the feedback device 20 can be an audio and/or visual feedback device including speakers 22 for emitting auditory alerts and a visual display 24 for displaying visual indications representing alerts, alarms, and other information. In some examples, the controller 14 can also be configured to display an indication, such as numerical representations, of the concentration of the VOC on the visual display 24 of the audio/visual feedback device 20. For example, the controller 14 can be configured to display real-time VOC concentration values on the visual display 24. The controller 14 can also be configured to display historical VOC values on the visual display 14 so that the user can appreciate changes in VOC levels over time for a particular location. The feedback device 20 can also include various arrangements of light emitters (e.g., light emitting diodes (LEDs) or other cables or bulbs) configured to illuminate to convey alerts and/or other information about environmental conditions or about the status of the VOC sensors 12 to the user or bystanders.

In some examples, the system 10 also includes a wireless transmitter 26 electrically coupled to the controller 14. The controller 14 can be configured to cause the wireless transmitter 26 to transmit alarms and alerts generated by the controller 14 to a remote device, system, or computer network. For example, the controller 14 can be configured to transmit alarms and alerts to other computer devices within a medical facility to alert others in the facility about possible gas leaks or unsafe levels of VOCs. The wireless transmitter 26 can be a short-range wireless data transmitter, such as a BLUETOOTH® transmitter. In other examples, the wireless transmitter 26 can be a long-range transmitter that transmits data to computer devices, systems, and networks in other locations, such as a WiFi transmitter or cellular transmitter.

In some examples, the system controller 14 can be configured to perform additional processing steps to increase accuracy of VOC detection and identification. For example, the controller 14 can be configured to normalize signals received from the VOC sensors 12 to account for environmental conditions. In some examples, processing signals received from the VOC sensor 12 can include normalizing the received signals for humidity or temperature based on information sensed by the humidity sensor 16 and/or temperature sensor 18. Processing received signals can also include modifying the received signals to account for portions of the signals and/or signal artifacts caused by chemicals or compounds other than a VOC of particular interest. For example, the controller 14 can be configured to distinguish between signals representative of a VOC of particular interest and signals representative of less hazardous chemicals (such as commonly used cleaning chemicals, including isopropyl alcohol, hydrogen peroxide, or bleach). The controller 14 can also be configured to remove artifacts in the received signals representative of chemicals other than a selected VOC of interest.

As previously described, the VOC sensor 12 can be any of a variety of different types of sensors that sense signals representative of VOCs in ambient air. For example, the VOC sensor 12 can include a metal oxide-based sensor, a sensor comprising graphene or a graphene derivative, a hot-wire anemometer, an infrared detector, or a biosensor. In some examples, metal-oxide gas sensors are based on a film of metal-oxide particles between two electrodes located on top of a heating element, such as a hotplate. Heating the metal oxide yields negatively charged oxygen species absorbed on the metal-oxide surface. The surface oxygen species react with ambient target gases and thereby release electrons into the metal-oxide film resulting in a change of electrical resistivity of the metal-oxide layer. The change of resistivity is measured between the two electrodes and directly depends on the ambient target gas concentration. An exemplary metal oxide-based sensor that can be used with the system 10 of the present disclosure is the Bosch BME688 gas sensor manufactured by Bosch Sensortec Gmbh.

In other examples, the VOC sensors 12 can comprise infra-red detectors, which rely on the fact that gas molecules absorb specific band of IR light. A common approach for IR detection is based on the irradiation of the confined gaseous compounds by light and the acquisition of their spectral information. Infrared spectroscopy relies on the fact that molecules absorb specific frequencies that are characteristic of their structure. These absorptions are resonant frequencies, i.e., the frequency of the absorbed radiation matches the frequency of the bond or group that vibrates. The energies are determined by the shape of the molecular potential energy surfaces, the masses of the atoms, and the associated vibronic coupling.

In a similar manner, Fourier Transform InfraRed (FTIR) gas analyzers and sensors identify and measure gaseous compounds by the compound's absorbance of infrared radiation. This detection scheme is possible because the combination of atoms and their arrangement is unique to every molecular structure. Therefore, molecules produce a unique spectrum when exposed to infrared light. Instrumental analysis of the spectrum gathered from infrared with wavelength around 2-12 micrometers enables the qualitative identification and quantitative analysis of the gaseous compounds in the sample gas. Exemplary FTIR based sensors that can be used with the systems 10 and devices of the present disclosure include sensors manufactured by Gasmet Technologies Oy of Vantaa, Finland.

Photoacoustic Spectroscopy (PAS) gas detectors, such as detectors made by Innova®, can also be used with the systems 10 of the present disclosure. The PAS detectors directly measure the absorption of IR radiation by VOC molecules, which is proportional to concentration. More specifically, in a PAS instrument, the gas to be measured is irradiated by modulated infrared light of a pre-selected wavelength. The gas molecules absorb some of the light energy and convert it into an acoustic signal which is detected by microphones.

Graphene or graphene derivative-based sensors, such as sensors by Arborsense, Inc., can also be used with the systems 10 of the present disclosure. The graphene based sensors include graphene reactive layers that change in properties, such as electrical conductivity, upon interaction with VOCs. For example, changes in a dipole moment of graphene molecules can be detected and used to identify different VOCs.

Biosensors with peptide molecules sensitive to and which change their measurable properties upon interaction with VOCs, such as the NeOse Advance sensors made by Aryballe Technologies, can also be used with the systems 10 and devices of the present disclosure. For example, the VOC sensor 12 can comprise a silicon substrate with an array of biosensors spotted on a surface of the silicon substrate configured to detect different VOCs by measuring changes in optical properties of peptides upon interaction with VOCs. The signal detected from an odor depends on the interaction between VOCs from the samples and the different biosensors of the sensor array.

In other examples, the VOC sensor 12 can comprise a gas flow and VOC sensor, such as sensors made by Sensirion AG., which are based on hot-wire anemometers. Hot-wire anemometer sensors have been used for air flow measurements in respiratory applications. The hot-wire anemometer sensors comprise a thin wire placed in the gas stream and heated. By measuring the heat loss of the wire, the velocity or flow of the air flow stream can be determined. Such hot-wire anemometers are typically analog devices, which can be sensitive to shock and vibration, and which age over time. In some examples, sensors including hot-wire anemometers may require frequent re-calibration., making such sensors unsuitable for use in applications where recalibration is difficult to perform.

### Stand-Alone VOC Detector Devices

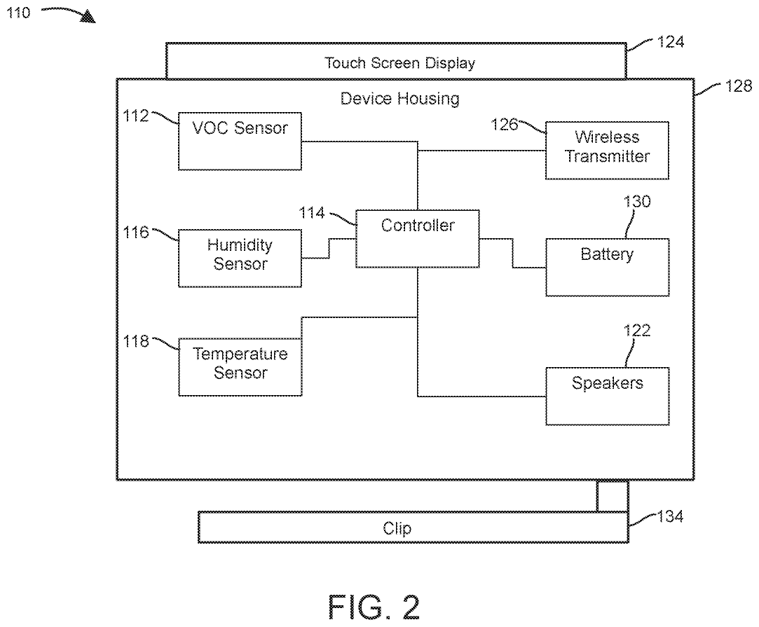

With reference to FIG. 2, in some examples, the VOC detector is a stand-alone detector or device 110, such as a portable and/or wearable device, that can be carried by a medical professional during performance of a medical procedure. In other examples, a user can attach the stand-alone device 110 to an object or medical device in a medical setting to obtain VOC measurements and/or to provide alarms when VOC concentration exceeds threshold values. For example, the stand-alone detector or device 110 can be configured to be mounted or attached to a surgical/patient bed, surgical curtain or drape, medical cart, or to a medical device, such as an anesthesia machine.

In some examples, the device 110 includes a housing 128, such as a plastic housing sized to be carried by the user. The VOC sensor 112, controller 114, and other electrical components of the detector device 110, such as a battery 130 or power source, speakers 122, computer readable memory 132, and the wireless transmitter 126, can be positioned within and/or enclosed by the housing 128. In some examples, the detector device 110 also includes a visual display 124 (e.g., a capacitive touch screen display) positioned in and viewable through an opening in the housing 128. In some examples, the VOC sensor 112 is mounted to an exterior of the housing 128. Alternatively, the VOC sensor 112 can be positioned in the housing 128 and may include an air inlet or opening extending through the housing 128 so that ambient air can pass through the opening to the VOC sensor 112.

The stand-alone VOC detector device 110 can also include a clip 134 or fastener for mounting the device 110 to one of the objects or devices of the medical facility. For example, the clip 134 can be configured to secure the stand-alone device 110 to a rail of a patient bed, operating table, or to another object in an operating room or surgical theater, such as a drape or curtain. The clip 134 can also be used to secure the stand-only device 110 to other medical devices, stands, carts, or frames in proximity to a patient, workspace, or VOC gas source. In some examples, the clip 134 can also be used for mounting the device 110 to a side of a ventilation hood or a similar workstation used by pharmacists to prepare medications. In other examples, the device 110 can include straps, bands, brackets, adhesives, or other mounting devices or connectors for securing the stand-alone device 110 to an object at a medical facility or medical scene.

### Wearable VOC Detector Device

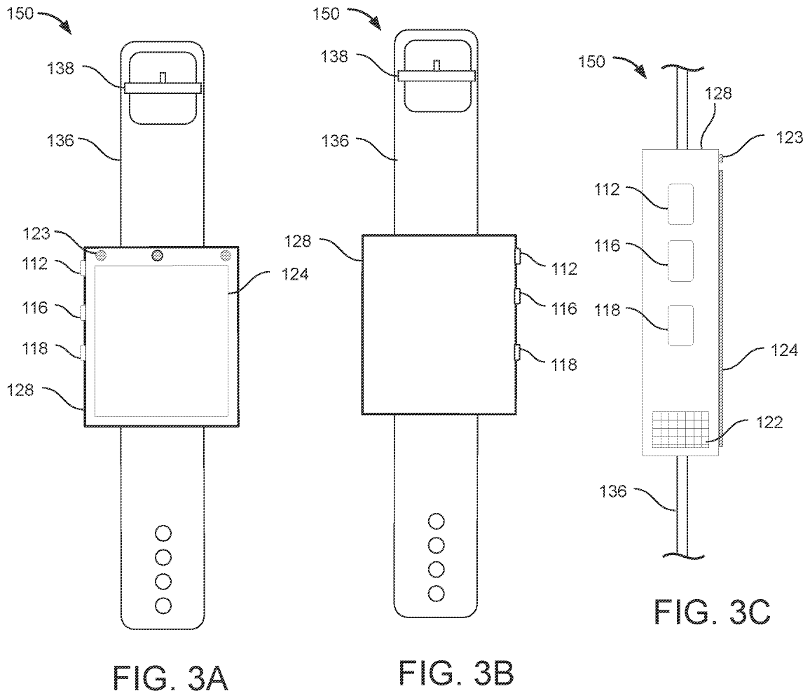

With reference to FIGS. 2 and 3A-3C, in some examples, the VOC detector device 110 is a wearable device 150 configured to be worn by a medical professional as he or she performs medical treatments and other duties for patients. For example, as previously described, the wearable device 150 can be a watch, bracelet, necklace, pendant, or similar wearable item.

An exemplary wearable device 150, such as a smart watch, for detecting VOCs is shown in FIGS. 3A-3C. The wearable device 150 or smart watch comprises the housing 128 containing the controller 114 (shown in FIG. 2), as well as other electronic components such as the computer memory 132, speakers 122 for emitting alarms, alerts, and warnings, a power supply or battery 130, and the visual display 124 or touch screen. The housing 128 can also include light sources or bulbs 123, such as light emitting diodes (LEDs), which can illuminate to convey alarms and other information about the wearable device 150 to a wearer. The housing 128 can be a size and shape of a conventional smart watch, such as a circle having a diameter of about 30 mm to about 50 mm or a rectangle with a length of about 30 mm to about 50 mm. As in previous examples, the VOC sensor 112 is electrically connected to the controller 114 and can be enclosed within or mounted to the housing 128. If the VOC sensor 112 is enclosed within the housing 128, the housing 128 can include an air inlet or opening for allowing ambient air to contact the reactive layers or portions of the sensor 112. As in previous examples, the controller 114 can cause alarms, alerts, or notifications to be displayed on the visual display 124 of the wearable device 150 when a measured concentration value for a VOC exceeds a threshold value. The controller 114 can also cause quantitative indications, such as numerical values for a sensed concentration of one or more VOCs, to be displayed on the visual display 124. As shown in FIGS. 3A-3C, the wearable device 150 or smart watch also includes a band 136 and clasp 138 for securing the wearable device 150 to a wearer's wrist.

As previously described, the wearable device 150 or watch is intended to be worn by a medical professional while performing actions in proximity to sources of VOCs. For example, a surgeon, anesthesiologist, or another member of an operating room team may wear the wearable device 150, while performing a procedure for an anesthetized patient, to determine if VOC molecules from anesthesia agents being administered to the patient are accumulating in ambient air in the operating room. In another example, a pharmacist may wear the wearable device 150 at a workstation while preparing, mixing, or reconstituting hazardous drugs that contain or off-gas VOCs.

In some examples, the wearable device 150 or watch can track a wearer's total exposure to one or more selected VOCs over a predetermined period of time, such as a shift, day, week, month, or year. For example, the controller 114 of the wearable device 150 can be configured to monitor the VOC concentration in ambient air for the selected period of time and display a graphical indication on the visual display 124 representative of the accumulated exposure. In some examples, the controller 114 can cause the display 124 to show a numerical value for the total exposure over the period of time. For example, the display 124 may show a numerical value for total volume of VOC gas inhaled by the wearer over the predetermined period of time. In other examples, a gauge (i.e., a gas gauge or similar icon) that moves between a first position and a second position over time may be displayed on the visual display 124. The second position of the gauge icon can represent a maximum permissible exposure for the wearer over the period of time. When the dial or similar icon reaches the second position, the controller 114 may cause the device 110 to display an alarm, alert, notification, or warning indicating that the wearer has reached a predetermined exposure level. The alarm, alert, notification, or warning may also include an instruction, shown in the visual display 124, stating that the wearer should move to another location away from the source of VOCs. Also, the alarm, alert, or notification may include an instruction to check VOC sources for leaks so that any leaks can be corrected, preventing or limiting further exposure to the VOCs.

In some examples, the wearable device 150 can also be configured to detect VOC concentration of gas exhaled by the wearer, such as a clinician or another medical professional, to assess the wearer's exposure to VOCs. For example, the controller 114 may cause the wearable device 150 to periodically emit requests that the wearer blow on the device 150. The controller 114 may process measurements by the VOC sensor 112 as the wearer is blowing on the device 150 to determine VOC concentration in the gas exhaled by the wearer. When the VOC concentration in the gas exhaled by the wearer exceeds a threshold value, the device 150 may issue an alarm or alert instructing the wearer to move away from the VOC source. For example, the threshold value may be a VOC concentration slightly below a concentration when the wearer begins to experience adverse effects of exposure to VOCs. The controller 114 may also track the VOC concentration in gas exhaled by the wearer over time to determine whether VOC concentration of gas exhaled by the wearer is increasing, decreasing, or remaining about the same. A detectable increase in VOC concentration of exhaled gas over time may indicate that VOC concentration in ambient air around the wearer is also increasing, which may mean that a VOC source is leaking or is otherwise in an unsafe condition.

### Anesthesia Machine With A VOC Detector

With reference to FIG. 4, in some examples, a VOC sensor 212 can be integrated in a medical device, such as an anesthesia machine 210, which is used to deliver medicated gasses, such as gasses comprising anesthesia agents containing VOCs, to and from a patient. As shown in FIG. 4, the anesthesia machine 210 can include a gas cylinder 240 containing the anesthesia agents to be delivered to the patient and a vaporizer 242 for creating an inhalable gas containing the anesthesia agents. The anesthesia machine 210 can also include a controller 214 for controlling components of the anesthesia machine 210 and a visual display 224 for displaying patient information and/or operating parameters of the machine 210.

The anesthesia machine 210 can also include or be connected to an air pathway, such as a delivery/breathing circuit 244, for delivering gasses from the gas cylinder 240 and a pump 274 or ventilator for moving air through the circuit 244. As shown schematically in FIG. 4, the delivery/breathing circuit 244 can be a conventional patient airway circuit comprising an inspiratory portion or limb 246 for transporting gas and an anesthesia agent from a gas inlet to the patient, an expiratory limb 248 for transporting exhaled air from the patient to an exhaust valve 250 and/or to cleaning portions 252 of the circuit 244, and a y-connector 254 for attaching the circuit 244 to a facemask 256 covering a nose and mouth of the patient. The anesthesia machine 210 can also include a gas pressure and/or flow regulator 258 connected to and/or fluidly connected to the inspiratory portion or limb 246 of the breathing circuit 244 for measuring gas flow through the circuit 244.

In some examples, the anesthesia machine 210 can also include multiple sensors for measuring physiological information about the patient, as well as for confirming that an appropriate amount of the anesthesia agent is being administered to the patient. The sensors can include, for example, a pressure sensor 260, airflow sensor 262, temperature sensor 264, oxygen sensor 266, and/or a capnography or CO₂ sensor 268 fluidly connected to the delivery/breathing circuit 244 for monitoring air inhaled or exhaled by the patient. As previously described, the anesthesia machine 210 can also include the cleaning portions 252 for cleaning and/or removing harmful molecules from air exhaled by the patient. In some examples, the cleaning portions 252 can include a canister or container 270 for absorbing carbon dioxide in exhaled air and a scrubber or scavenging device 272 for removing waste gasses from the exhaled air before the exhaled air passes through the exhaust valve 250 or returns to the inspiratory portion or limb 246 of the breathing circuit 244.

As previously described, the anesthesia machine 210 also includes the VOC sensor 212 integrated with other components of the anesthesia machine 210 and/or breathing circuit 244. In some examples, the VOC sensor 212 is positioned proximate to the return side or expiratory portion or limb 248 of the breathing circuit 244. For example, the VOC sensor 212 can be mounted to a housing of the machine 210 proximate to the exhaust valve 250 or to another port or relief valve fluidly connected to the expiratory portion or limb 248 of the anesthesia machine 210. As in previous examples, the VOC sensor 212 senses VOC molecules in ambient air, specifically ambient air in proximity to the exhaust valve 250 of the anesthesia machine 210, and provides signals representative of detected VOC molecules to a controller, such as the controller 214 of the anesthesia machine 210.

As previously described, the machine controller 214 can be configured to control the vaporizer 242, ventilator or pump 274, and other component of the anesthesia machine 210 for delivering the anesthesia agent to the patient. The anesthesia machine controller 214 is also configured to receive and process signals from the VOC sensor 212, determine a concentration of a selected VOC in ambient air proximate to the VOC sensor 212 based on the received and processed signals, compare the determined concentration of the VOC to a threshold value to determine when the detected concentration exceeds the threshold value, and provide an alarm when the detected concentration of the VOC exceeds the threshold value. For example, the alarm may be emitted from speakers 222 of the anesthesia machine 210 or displayed on the visual display 224 of the anesthesia machine 210. As in previous examples, quantitative measurements for VOC concentration values determined from information sensed by the VOC sensor 212 can also be shown on the visual display 224.

In some examples, the anesthesia machine controller 214 can also monitor ambient air in proximity to the breathing circuit exhaust valve 250 after a procedure has been completed to determine when anesthesia agents have dissipated from the patient's exhaled air and it is safe to remove the patient's facemask 256. For example, the anesthesia machine controller 214 can be configured to compare a determined concentration of a selected VOC in ambient air to a normal or baseline value for a patient prior to commencing anesthesia treatment. The normal value may be determined by measuring VOC concentration in a patient's exhaled air before the anesthesia agent is provided to the patient. In other examples, a normal or accepted baseline value determined by monitoring VOC levels in exhaled air from many patients could be used. Following the comparison, the controller 214 can be configured to provide an indication to a user that the patient's breathing has returned to normal when the concentration of the selected VOC is below or within a predetermined amount of the normal value for the patient. In some examples, the indication provided to the user can include an instruction to remove the facemask 256 from the patient's face.

### Sensor For VOC Detection

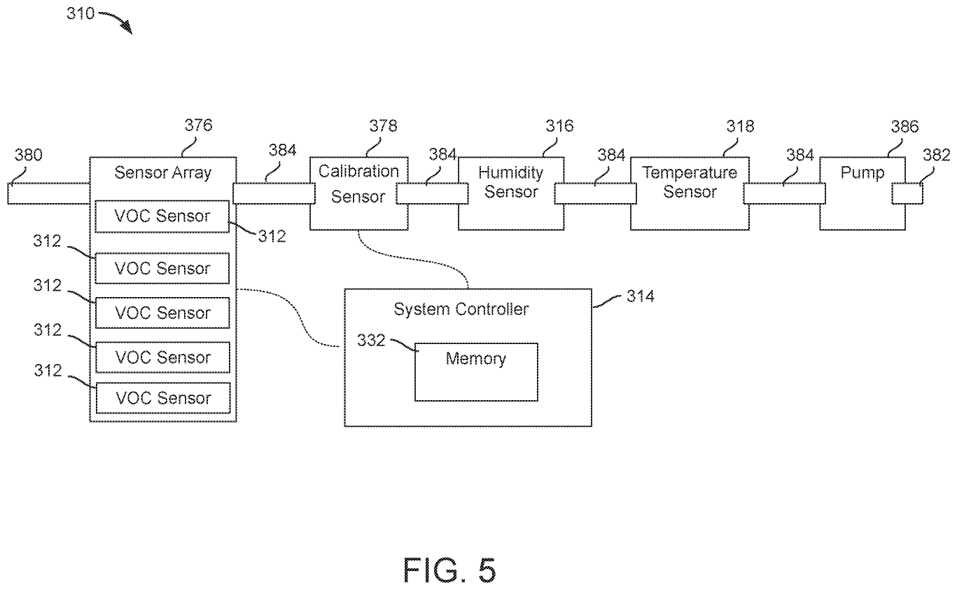

FIG. 5 shows another example of a VOC detection device 310 comprising a VOC sensor 312 and a controller 314. Unlike in previous examples, the VOC detection device 310 of FIG. 5 includes a sensor array 376 comprising a plurality of VOC sensors 312 configured to identify and detect concentrations of different VOCs. An exemplary sensor array comprising sensors for detecting VOCs, which can be used with the detection devices and systems of the present disclosure, is disclosed in U.S. Pat. No. 10,928,369, which is incorporated herein by reference in its entirety.

In some examples, the VOC sensors 312 of the sensor array 376 are configured to exhibit slightly different responses to the VOCs, such that the sensor array 376 generates a unique signature pattern for molecules or odors in ambient air in proximity to the sensor array 376. The generated signature can be compared to signatures for known VOCs to determine VOCs present in ambient air.

In some examples, the device 310 can further include computer memory 332 electrically connected to the controller 314. The memory 332 can contain previously-determined VOC signatures for a plurality of VOC compounds. In that case, the controller 314 can compare the signature generated by the sensor array 376 to the previously-determined signatures for known VOCs contained on the computer memory 332 to identify VOC compound(s) in ambient air proximate to the sensor array 376. The controller 314 can also be configured to update the VOC signature stored on the computer memory 332 for an identified VOC compound based on signals sensed by the VOC sensors 312 of the sensor array 376.

With continued reference to FIG. 5, the VOC detection device 310 can also include a second type of a VOC sensor 312, referred to herein as a calibration sensor 378, for calibrating sensors 312 of the sensor array 376 and/or for confirming that measurements from the sensor array 376 are accurate. For example, the calibration sensor 378 can be a metal-oxide based sensor, an infrared detector, and/or a photo-ionisation detector, or another type of sensor capable of sensing VOCs, configured to measure properties of the same ambient air that contacts the plurality of VOC sensors 312 of the sensor array 376. In other examples, the sensor array 376 can be calibrated using measurements from, for example, a gas chromatograph mass spectrometer.

As shown in FIG. 5, the sensor array 376 and the calibration sensor 378 can be fluidly connected together. For example, the device 310 can include an air inlet 380 and an air outlet 382 positioned such that ambient air passes into the inlet 380 and to the calibration sensor 378. The device 310 can further include a flow tube 384 defining an air pathway between the calibration sensor 378 and the sensors 312 of the sensor array 376. The device 310 can also include a pump or fan 386 fluidly connected to the flow tube 384 or air pathway configured to draw ambient air into and through the flow tube 384 or air pathway.

The calibration sensor 378 can be electrically connected to the controller 314. The controller 314 can be configured to receive measurements from both the calibration sensor 378 and the sensor array 376, as well as from a humidity sensor 316 and temperature sensor 318. The measurements can be compared to calibrate the sensors 312 of the array 376. In particular, if the controller 314 identifies a discrepancy between measurements of the calibration sensor 378 and signals detected by the sensor array 376, the controller 314 can be configured to automatically recalibrate the sensors 312 of the sensor array 376 to account for the identified discrepancy.

While examples of the gas sensing and detection systems and devices of the present disclosure are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

1. A system for detection of volatile organic compounds (VOCs) in a medical facility, the system comprising:
at least one VOC sensor configured to sense VOCs in ambient air of the medical facility; and
a controller electrically connected to the at least one VOC sensor configured to:
receive and process signals from the at least one VOC sensor;
determine a concentration of at least one VOC in ambient air proximate to the at least one VOC sensor based on the received and processed signals;
compare the determined concentration of the at least one VOC to a threshold value to determine when the detected concentration exceeds the threshold value; and
provide an alarm when the detected concentration of the at least one VOC exceeds the threshold value.

2. The system of claim 1, wherein the medical facility comprises at least one of a hospital, operating room, recovery room, intensive care unit, out-patient medical office, dental office, ambulance, or veterinary clinic.

3. The system of claim 1, wherein the at least one VOC sensed by the at least one VOC sensor comprises a halogenated hydrocarbon.

4. The system of claim 1, wherein the at least one VOC sensed by the at least one VOC sensor comprises at least one of sevoflurane, enflurane, desflurane, halothane, isoflurane, nitrous oxide, indole, methanol, undecene, ethylacetate, 2,4-dimethyl-1-heptane, butanone, benzaldehyde, dimethylcyclohexanol, isovaleric acid, 2-pentanol, methylquinazoline, or methyl butyraldehyde.

5. The system of claim 1, wherein the at least one VOC sensed by the at least one VOC sensor is a sterilization gas, such as ethylene oxide (EtO) or hydrogen peroxide.

6. The system of claim 1, wherein the at least one VOC sensed by the at least one VOC sensor comprises VOCs emitted from hazardous drugs to be delivered to a patient, such as chemotherapy drugs.

7. The system of claim 1, further comprising a housing, wherein the at least one VOC sensor and the controller are enclosed within and/or mounted to the housing.

8. The system of claim 7, wherein the housing comprises a clip for mounting the housing to at least one of a patient bed, surgical curtain or drape, medical cart or stand, medical device (e.g., anesthesia gas equipment), an anesthesia waste gas scavenger, pharmaceutical workstation, ventilated hood, or pharmaceutical storage area.

9. The system of claim 7, wherein the housing is a wearable housing or is configured to be mounted to a portable electronic device, such as a smart phone or computer tablet

10. The system of claim 1, further comprising an audio/visual feedback device electrically connected to the controller, wherein the controller is configured to cause the feedback device to emit the alarm to a user.

11. The system of claim 10, wherein the audio/visual feedback device comprises at least one of a speaker, visual display, or light emitter.

12. The system of claim 10, wherein the controller is further configured to cause the audio/visual feedback device to display an indication of the concentration of the at least one VOC on a visual display of the audio/visual feedback device.

13. The system of claim 12, wherein the indication comprises a numerical value for the determined concentration displayed on the visual display.

14. The system of claim 1, further comprising a wireless transmitter electrically coupled to the controller, wherein the controller is configured to cause the wireless transmitter to transmit the alarm and/or information sensed by the at least one VOC sensor to a remote device, system, or computer network.

15. The system of claim 1, wherein the at least one VOC sensor comprises at least one of a metal oxide-based sensor, a sensor comprising graphene or a graphene derivative, or an infrared detector.

16. (canceled)

17. The system of claim 1, wherein the at least one VOC sensor comprises a biosensor configured to detect peptide molecules based on optical properties of the molecules.

18. The system of claim 1, further comprising a humidity and/or temperature sensor, and wherein processing the signals from the at least one VOC sensor comprises normalizing the received signals for at least one of humidity or temperature based at least in part in information sensed by the humidity and/or temperature sensor.

19. (canceled)

20. The system of claim 1, wherein processing the received signals comprises distinguishing between signals representative of the at least one VOC and signals representative of cleaning chemicals (such as isopropyl alcohol, hydrogen peroxide, or bleach) used during medical procedures.

21. (canceled)

22. The system of claim 1, further comprising an anesthesia machine, wherein the at least one VOC sensor is positioned to detect ambient air proximate to an exhalation portion of the anesthesia machine, wherein the anesthesia machine comprises:
at least one gas cylinder,
at least one gas pressure and/or flow regulator,
a vaporizer,
a delivery/breathing circuit,
at least one of a pressure sensor, airflow sensor, temperature sensor, oxygen sensor, or CO₂ sensor fluidly connected to the delivery/breathing circuit for monitoring air inhaled or exhaled by a patient,
a scavenging system, and
a face mask.

23. (canceled)

24. The system of claim 22, wherein the controller is further configured to compare the determined concentration of the at least one VOC to a normal value for the patient prior to commencing anesthesia treatment; and
provide an indication that the patient's breathing has returned to normal when the concentration is below or within a predetermined amount of the normal value for the patient.

25-32. (canceled)
